(19) United States
(12) Patent Application Publication
(10) Pub. No.: US 2023/0236203 A1
(43) Pub. Date: Jul. 27, 2023

(54) Method for Determining Risk of Pre-Term Birth

(71) Applicants: Board of Regents, The University of Texas System, Austin, TX (US); Arcedi Biotech ApS, Vejle (DK)

(72) Inventors: Ramkumar Menon, Galveston, TX (US); Ripudaman Singh, Aarhus (DK); Palle Schelde Jensen, Aarhus (DK); Inga Baasch Christensen, Risskov (DK); Lotte Hatt, Skanderborg (DK); Katarina Kiilerich Ravn, Hoebjerg (DK)

(21) Appl. No.: 18/009,576

(22) PCT Filed: Jun. 9, 2021
(86) PCT No.: PCT/US2021/036562

§ 371 Date: Dec. 9, 2022

Related U.S. Application Data
(60) Provisional application No. 63/037,212, filed on Jun. 10, 2020.

(51) Int. Cl. G01N 33/68

(52) U.S. Cl. CPC G01N 33/689; G01N 2800/368; G01N 2800/50

(57) ABSTRACT

The present invention includes a method for determining pre-term birth or an associated clinical condition or an increased risk of pre-term birth or an associated clinical condition based on the presence and/or increased amount of specific amnion and/or chorion cells in a blood sample from a pregnant woman.

### CROSS-REFERENCE TO RELATED APPLICATIONS

This application claims the benefit of U.S. Provisional Appl. No. 63/037,212, filed Jun. 10, 2020. The entire content of which are hereby incorporated by reference.

### TECHNICAL FIELD OF THE INVENTION

The present invention relates to a method for determining pre-term birth, an associated clinical condition, an increased risk of pre-term birth, and/or an associated clinical condition based on the presence and/or increased amount of specific amnion and/or chorion cells in a blood sample from a pregnant woman.

### STATEMENT OF FEDERALLY FUNDED RESEARCH

None.

### BACKGROUND OF THE INVENTION

Without limiting the scope of the invention, its background is described in connection with pre-term birth (PTB).

Pre-term birth, also known as premature birth, is the birth of a baby before 37+0 weeks gestational age. The earliest gestational age at which the infant has at least a 50% chance of survival is referred to as the limit of viability. As care of pre-term infants has improved, the limit of viability has reduced to approximately 24 weeks gestational age. However, pre-term birth is associated with a markedly increased risk of disabilities of the infant, and the risk is higher the lower the gestational age. It is therefore highly important to delay/prevent the birth/labor from a pregnant woman being at increased risk of pre-term birth as long as possible.

The exact cause of preterm birth is difficult to determine and it may be multi-factorial. Risk factors that have been identified as increasing the risk of pre-term birth include: diabetes, high blood pressure, being pregnant with more than one baby, being either obese or underweight, a number of vaginal infections, tobacco smoking, and psychological stress, among others.

The complications associated with pre-term birth include cerebral palsy, delays in development, hearing problems, and sight problems among others. In the normal human fetus, several organ systems mature between 34 and 37 gestational weeks, and the fetus reaches adequate maturity by the end of this period. One of the main organs greatly affected by pre-term birth is the lungs. The lungs are one of the last organs to mature in the womb and because of this, many premature babies spend the first days and weeks of their lives on ventilators. Preterm babies born near 37 weeks of gestational age often have no problems relating to prematurity if their lungs have developed adequate surfactant, which allows the lungs to remain expanded between breaths. Sequelae of prematurity can be reduced to a small extent by using drugs to accelerate maturation of the fetus, however, it can be reduced to an even greater extent by delaying the birth or preventing preterm birth altogether. In this aspect, it is critical to be able to identify pregnant women at (increased) risk of pre-term birth to be able to provide treatment.

One such patent is U.S. Pat. No. 10,240,199, issued to Lo, et al., entitled “Maternal plasma transcriptome analysis by massively parallel RNA sequencing.” These inventors are said to teach methods for diagnosing pregnancy-associated disorders, determining allelic ratios, determining maternal or fetal contributions to circulating transcripts, and/or identifying maternal or fetal markers using a sample from a pregnant female subject, and the use of a gene for diagnosing a pregnancy-associated disorder in a pregnant female subject.

Another such patent is U.S. Pat. No. 9,417,249, issued to Taylor, et al., entitled “Methods of predicting and decreasing the risk of pre-term birth.” These inventors are said to teach methods for predicting the risk of pre-term birth in a pregnant subject, for identifying a subject having an increased risk of pre-term birth, for selecting a subject for participation in a clinical study, and for decreasing the risk of pre-term birth in a subject. These methods include providing a sample from the subject and detecting the level of one or more of growth arrest-specific protein 1 (GASI), ALLI-fused gene from chromosome 4 protein (AR4)/Fragile X Mental Retardation 2 (FMR2) family member 3 (AFF3), transthyretin (TTR), ryanodine receptor 1 (RYRI), E26 transformation specific variant 6 (ETV6), claudin-10, zinc finger protein 23 (ZNF23), collagen type XXVII al (COL27AI), Kazrin isoform-1, keratin-associated protein 10-9 (KRTAPIO-9), Huntingtin (HTT), microtubule associated protein 9 (MAPS), coiled-coil domain-containing protein 13 (CCDC13), inositol hexakisphosphate and diphosphoinositol-pentakisphosphate kinase isoform 2 (HISPPDI), immunoglobulin gamma-3 chain C (IGHG3), cysteine- and histidine-rich protein-1 (CYHRI), and XP 002348181

However, despite these advances, what is needed are robust methods and markers for identifying patients that are likely to develop complications during pregnancy.

### SUMMARY OF THE INVENTION

The present invention relates to the above-described important issue of identifying pregnant women at increased risk of pre-term birth. This is crucial in order to either delay the birth or prevent the pre-term birth, or alternatively to provide the specialized care needed to limit the complications associated with pre-term birth.

Amniochorionic membranes play a major role during gestation and parturition. Microfractures in the fetal membranes are one of the processes that lead to human parturition. The inventors have surprisingly identified a correlation between specific amnion and/or chorion cells in the blood of pregnant women at increased risk of pre-term birth or that have a preterm or premature rupture of membranes. Microfractures in the fetal membranes may cause invasion into maternal blood with cells from these membranes. Hence, early detection of these cells in maternal blood can be used to identify pregnancies that are at risk of pre-term birth.

Based on this finding, the inventors have developed a method for determining pre-term birth or an increased risk of pre-term birth based on the presence and/or increased amount of said amnion and/or chorion cells in a blood sample of said pregnant woman.

In one embodiment, the present invention includes a method of determining pre-term birth or an associated clinical condition or an increased risk of pre-term birth or an associated clinical condition, the method comprising the steps of: (a) providing a blood sample or a fraction thereof isolated from a pregnant woman, and (b) determining the presence or characteristics of amnion and/or chorion cells in the blood sample or fraction thereof; wherein the presence or characteristics of amnion and/or chorion cells is indicative of pre-term birth or an associated clinical condition or an increased risk of pre-term birth or an associated clinical condition of the pregnant woman. In one aspect, the presence of amnion and/or chorion cells is determined by detecting one or more specific amnion and/or chorion cell markers. In another aspect, the specific amnion and/or chorion cell markers are differentially expressed in amnion and/or chorion cells compared to a blood sample or a fraction thereof isolated from a pregnant woman. In another aspect, the log 2 fold difference between the expression of the amnion and/or chorion cell marker in amnion and/or chorion cells compared with the expression in a blood sample or fraction thereof is at least 5, at least 10, or at least 15. In another aspect, the method comprises the steps of: (a) providing a blood sample or a fraction thereof isolated from a pregnant woman, (b) contacting the sample with (i) a ligand directed to an amnion and/or chorion cell marker or (ii) a hybridization probe comprising at least 10 contiguous nucleotides complementary to a gene encoding the amnion and/or chorion cell marker, and (c) detecting the amnion and/or chorion cell marker in the blood sample or fraction thereof of (a); wherein the presence of an amnion and/or chorion cell marker is indicative of pre-term birth or an associated clinical condition or an increased risk of pre-term birth or an associated clinical condition of the pregnant woman. In another aspect, the amnion and/or chorion cell marker is selected from the group consisting of IGF2, IGFPB3, SERPINB10, FBN1, CRYAB, KRT18, PRTG, COL1A2, GPX8, MUC16, KRT5, TSPAN1, UPK1B, CADPS2, LAMC2, AHNAK2, EMP1, FN1, MET, PVRL4, A2ML1, DSP, THSD4, KRT17, PDLIM4, COL17A1, DKK3, PLS3, DPYSL3, TPPP3, SHROOM3, THY1, DCN, DIO2, IGFBP2, SPARCL1, NNMT, LPHN3, PEG3, FLT1, NPR3, AOC1, ITGB8, RXFP1, SPOCK1, CYP11A1, COL4A2, CNR1, SEMA3A, SERPINE1, IL1R1, FBLN1, UCHL1, RAI2, TGM2, PRLR, FSTL3, BCAR1, THSD4, FERMT2, PKP2, P4HA2, TEAD1, and AQPEP. In another aspect, the amnion and/or chorion cell marker is selected from the group consisting of MUC16, UPK1B, EMP1, PVRL4, THY1, DIO2, LPHN3, FLT1, NPR3, RXFP1, CNR1, PRLR, IGF2, IGFBP3, SERPINB10, FBN1, CRYAB, KRT18, PRTG, COL1A2 and GPX8. In another aspect, the amnion and/or chorion cell marker is selected from the group consisting of MUC16, UPK1B, EMP1, PVRL4, THY1, DIO2, LPHN3, FLT1, NPR3, RXFP1, CNR1 and PRLR. In another aspect, the amnion and/or chorion cell marker is selected from the group consisting of MUC16, UPK1B, EMP1 and PVRL4. In another aspect, the amnion and/or chorion cell marker is selected from the group consisting of THY1, DIO2, LPHN3, FLT1, NPR3, RXFP1, CNR1 and PRLR. In another aspect, the amnion and/or chorion cell marker is selected from the group consisting of IGF2, IGFBP3, SERPINB10, FBN1, CRYAB, KRT18, PRTG, COL1A2 and GPX8. In another aspect, the amnion and/or chorion cell marker is selected from the group consisting of MUC16, UPK1B, EMP1, GPX8, FLT1/VEGFR1, RXFP1, CNR1 and PRLR. In another aspect, the method further comprises detecting an epithelial marker, such as a marker selected from the group consisting of CK1, CK2, CK3, CK4, CK5, CK6, CK7, CK8, CK9, CK10, CK12, CK13, CK14, CK15, CK16, CK17, CK18, and CK19. In another aspect, the method further comprises detecting a maternal marker such as a marker selected from the group consisting of CD14 and CD45. In another aspect, the method further comprises detecting a mesenchymal marker, such as a marker selected from at least one of Vimentin. In another aspect, the clinical condition associated with pre-term birth is preeclampsia. In another aspect, the method further comprises: determining the amount of amnion and/or chorion cells in the blood sample or fraction thereof, and comparing the amount of amnion and/or chorion cells to a control; wherein an amount of amnion and/or chorion cells in the blood sample or fraction thereof higher than the amount in a control is indicative of pre-term birth or an associated clinical condition or an increased risk of pre-term birth or an associated clinical condition of the pregnant woman. In another aspect, the amount of amnion and/or chorion cells in a control is a pre-determined value. In another aspect, the method further comprises: determining the amount of amnion and/or chorion cell markers in the blood sample or fraction thereof, and comparing the amount of amnion and/or chorion cell marker to a control; wherein an amount of amnion and/or chorion cell markers in the blood sample or fraction thereof higher than the amount in a control is indicative of pre-term birth or an associated clinical condition or an increased risk of pre-term birth or an associated clinical condition of the pregnant woman. In another aspect, the amount of amnion and/or chorion cell marker in a control is a pre-determined value. In another aspect, the amnion and/or chorion cell marker is detected at the protein level and/or the RNA level. In another aspect, the blood sample is isolated from the pregnant woman after 20 weeks of gestation. In another aspect, the amnion and/or chorion cell marker is present in the cell membrane and/or are intracellular. In another aspect, the blood sample from a pregnant woman is whole blood. In another aspect, wherein a treatment is provided to the pregnant woman for minimizing the risk of preterm birth or ameliorating the consequences of pre-term birth, such as hospitalizing the individual.

In another embodiment, the present invention includes an assay for identifying fetal markers in a blood sample obtained from a pregnant woman comprising: obtaining the blood sample from the pregnant woman; and determining the presence of one or more biomarkers selected from IGF2, IGFPB3, SERPINB10, FBN1, CRYAB, KRT18, PRTG, COL1A2, GPX8, MUC16, KRT5, TSPAN1, UPK1B, CADPS2, LAMC2, AHNAK2, EMP1, FN1, MET, PVRL4, A2ML1, DSP, THSD4, KRT17, PDLIM4, COL17A1, DKK3, PLS3, DPYSL3, TPPP3, SHROOM3, THY1, DCN, DIO2, IGFBP2, SPARCL1, NNMT, LPHN3, PEG3, FLT1, NPR3, AOC1, ITGB8, RXFP1, SPOCK1, CYP11A1, COL4A2, CNR1, SEMA3A, SERPINE1, IL1R1, FBLN1, UCHL1, RAI2, TGM2, PRLR, FSTL3, BCAR1, THSD4, FERMT2, PKP2, P4HA2, TEAD1, or AQPEP, in amnion and/or chorion cells in the blood sample or fraction thereof. In another embodiment, the present invention includes an assay for identifying fetal markers in a blood sample obtained from a pregnant woman comprising: determining the presence of one or more biomarkers in a blood sample obtained from the pregnant woman, wherein the one or more biomarkers are selected from IGF2, IGFPB3, SERPINB10, FBN1, CRYAB, KRT18, PRTG, COL1A2, GPX8, MUC16, KRT5, TSPAN1, UPK1B, CADPS2, LAMC2, AHNAK2, EMP1, FN1, MET, PVRL4, A2ML1, DSP, THSD4, KRT17, PDLIM4, COL17A1, DKK3, PLS3, DPYSL3, TPPP3, SHROOM3, THY1, DCN, DIO2, IGFBP2, SPARCL1, NNMT, LPHN3, PEG3, FLT1, NPR3, AOC1, ITGB8, RXFP1, SPOCK1, CYP11A1, COL4A2, CNR1, SEMA3A, SERPINE1, IL1R1, FBLN1, UCHL1, RAI2, TGM2, PRLR, FSTL3, BCAR1, THSD4, FERMT2, PKP2, P4HA2, TEAD1, or AQPEP, in amnion and/or chorion cells in the blood sample or fraction thereof. In one aspect, the presence of amnion and/or chorion cells is indicative of pre-term birth or an associated clinical condition or an increased risk of pre-term birth or an associated clinical condition of the pregnant woman.

In another embodiment, the present invention includes a method of identifying a patient in need of decreased physical activity and/or bed rest during pregnancy comprising: performing an assay for identifying fetal markers in a blood sample obtained from the patient comprising: obtaining the blood sample from the patient; and determining the presence of one or more biomarkers selected from IGF2, IGFPB3, SERPINB10, FBN1, CRYAB, KRT18, PRTG, COL1A2, GPX8, MUC16, KRT5, TSPAN1, UPK1B, CADPS2, LAMC2, AHNAK2, EMP1, FN1, MET, PVRL4, A2ML1, DSP, THSD4, KRT17, PDLIM4, COL17A1, DKK3, PLS3, DPYSL3, TPPP3, SHROOM3, THY1, DCN, DIO2, IGFBP2, SPARCL1, NNMT, LPHN3, PEG3, FLT1, NPR3, AOC1, ITGB8, RXFP1, SPOCK1, CYP11A1, COL4A2, CNR1, SEMA3A, SERPINE1, IL1R1, FBLN1, UCHL1, RAI2, TGM2, PRLR, FSTL3, BCAR1, THSD4, FERMT2, PKP2, P4HA2, TEAD1, or AQPEP, in amnion and/or chorion cells in the blood sample or fraction thereof, wherein the presence of amnion and/or chorion cells with one or more of the biomarkers is indicative of pre-term birth or an associated clinical condition or an increased risk of pre-term birth or an associated clinical condition of the pregnant woman; and directing the patient to reduce physical activities and/or bed rest. In another embodiment, the present invention includes a method of identifying a patient in need of decreased physical activity and/or bed rest during pregnancy comprising: performing an assay for identifying fetal markers in a blood sample obtained from the patient comprising: determining the presence of one or more biomarkers in a blood sample obtained sample from the patient, wherein the one or more biomarkers are selected from IGF2, IGFPB3, SERPINB10, FBN1, CRYAB, KRT18, PRTG, COL1A2, GPX8, MUC16, KRT5, TSPAN1, UPK1B, CADPS2, LAMC2, AHNAK2, EMP1, FN1, MET, PVRL4, A2ML1, DSP, THSD4, KRT17, PDLIM4, COL17A1, DKK3, PLS3, DPYSL3, TPPP3, SHROOM3, THY1, DCN, DIO2, IGFBP2, SPARCL1, NNMT, LPHN3, PEG3, FLT1, NPR3, AOC1, ITGB8, RXFP1, SPOCK1, CYP11A1, COL4A2, CNR1, SEMA3A, SERPINE1, IL1R1, FBLN1, UCHL1, RAI2, TGM2, PRLR, FSTL3, BCAR1, THSD4, FERMT2, PKP2, P4HA2, TEAD1, or AQPEP, in amnion and/or chorion cells in the blood sample or fraction thereof, wherein the presence of amnion and/or chorion cells with one or more of the biomarkers is indicative of pre-term birth or an associated clinical condition or an increased risk of pre-term birth or an associated clinical condition of the pregnant woman; and directing the patient to reduce physical activities and/or bed rest. In one aspect, the amnion and/or chorion cell marker is detected at the protein level and/or the RNA level. In another aspect, the blood sample is isolated from the pregnant woman after 20 weeks of gestation. In another aspect, the amnion and/or chorion cell marker is present in the cell membrane. In another aspect, the blood sample from a pregnant woman is whole blood.

A kit comprising one or more agents for the detection of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 49, 49, 50, 51, 52, 53, 54, 55, 56, 57, 58, 59, 60, 61, 62, 63 or all the markers in Table 1, on amnion and/or chorion cells in a blood sample or fraction thereof.

### BRIEF DESCRIPTION OF THE DRAWINGS

For a more complete understanding of the features and advantages of the present invention, reference is now made to the detailed description of the invention along with the accompanying figures and in which:

FIG. 1 summarizes the steps for isolating fetal membrane cells from maternal blood samples of the present invention.

FIG. 2A shows fetal membrane candidate cells identified in maternal blood using antibodies specific for fetal membrane cells. FIG. 2B shows the same cells confirmed by X-Y FISH.

FIGS. 3A and 3B shows the expression of membrane specific markers in human primary amnion epithelia cells (AEC). Immunostaining shows AEC co-express epithelial marker cytokeratin-18 and mesenchymal marker vimentin, while “membrane” markers are seen on the cell and nuclear membrane and cytoplasm. FIG. 3A shows AEC express all three membrane specific markers in green, and FIG. 3B shows an additional five membrane markers in red. Fluorescent images were captured at 20×. These figures show one representative image from three separate experiments.

FIG. 4 shows the expression of membrane specific markers in human primary chorion trophoblast (CTC). Immunostaining shows CTC dominantly express epithelial marker cytokeratin-18 (green) but not mesenchymal marker vimentin, while “membrane” markers are seen in red. Fluorescent microscopy shows CTC express all eight membrane specific markers. Fluorescent images were captured at 20×. Blue—DAPI, red—chorion membrane marker of interest, yellow-vimentin, and green—Cytokeratin-18. This figure shows one representative image from three separate experiments.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present invention relates to a method for determining/predicting pre-term birth or an increased risk of pre-term birth from a blood sample from a pregnant woman. The method relies on the finding that defined amnion and/or chorion cells, detectable by specific cell markers, are present at an increased amount in the blood of a pregnant woman about to give birth. The presence of these cells, therefore, serves as an early marker of pre-term birth as well as clinical conditions associated with pre-term birth, such as preeclampsia.

Amniochorionic membranes play major role during gestation and parturition, and microfractures in the fetal membranes are one of the processes that lead to human parturition. The presently provided methodology for determining pre-term birth and risk thereof is based on the realization that the disruption of amniochorionic membranes also causes an invasion of maternal blood with the cells from these membranes. Hence, early detection of these cells in maternal blood can be used to identify pregnancies that are at risk of pre-term birth.

The present invention provides an important tool for identification of such pregnant women at increased risk of giving birth prematurely, thereby allowing for treatment of the pregnant woman for minimizing the risk of pre-term birth or ameliorating the consequences of pre-term birth, such as hospitalizing the individual.

Method of Determining Pre-Term Birth. Amnion and/or Chorion Cells.

In one embodiment, a method of determining/predicting pre-term birth or an associated clinical condition or an increased risk of pre-term birth or an associated clinical condition is provided, the method comprising the steps of: (a) providing a blood sample or a fraction thereof isolated from a pregnant woman, and (b) determining the presence of amnion and/or chorion cells in the blood sample or fraction thereof; wherein the presence of amnion and/or chorion cells is indicative of pre-term birth or an associated clinical condition or an increased risk of pre-term birth or an associated clinical condition of the pregnant woman.

In one embodiment, a method of determining/predicting pre-term birth or an associated clinical condition or an increased risk of pre-term birth or an associated clinical condition is provided, the method comprising the steps of: (a) determining the presence of amnion and/or chorion cells in a blood sample from a pregnant woman or fraction thereof; wherein the presence of amnion and/or chorion cells is indicative of pre-term birth or an associated clinical condition or an increased risk of pre-term birth or an associated clinical condition of the pregnant woman.

In one embodiment, the method further comprises: (a) determining the amount of amnion and/or chorion cells in the blood sample or fraction thereof, and (b) comparing the amount of amnion and/or chorion cells to a control, wherein an amount of amnion and/or chorion cells in the blood sample or fraction thereof higher than the amount in a control is indicative of pre-term birth or an associated clinical condition or an increased risk of pre-term birth or an associated clinical condition of the pregnant woman.

The amount of amnion and/or chorion cells in a control may be a pre-determined value. In one embodiment, the amount of amnion and/or chorion cells in a control is the amount of amnion and/or chorion cells in the blood of a pregnant woman not at risk of pre-term birth. In one embodiment, the amount of amnion and/or chorion cells in a control is a pre-determined value based on an average amount of amnion and/or chorion cells in the blood of a group of pregnant women not at risk of pre-term birth.

As used herein, the term “amnion” refers to the membrane that covers the embryo when first formed. It further contains the amniotic fluid and serves to provide a protective environment for the developing embryo or fetus.

As used herein, the term “cell-marker” refers to cell surface markers and intracellular markers, which are proteins expressed on the surface of cells and intracellularly and serve as markers of specific cell types. Cell markers play a role in inter-cellular and intra-cellular communication and recognition. Cell markers are specific to each kind of cell and may therefore serve as tools for identification of cells based on the cell markers present on the membrane of the cell and intracellularly.

As used herein, the term “chorion” refers to the outermost fetal membrane around the embryo. The chorion consists of two layers: an outer formed by the trophoblast, and an inner formed by the somatic mesoderm; the inner layer is in contact with the amnion.

As used herein, the term “hybridization probe” refers to a nucleic acid sequence capable of hybridizing with a gene encoding a given amnion and/or chorion cell marker. The hybridization probe comprises nucleotides complementary to the gene. The hybridization probe may be labeled with a reporter dye. Upon hybridization of the hybridization probe with the gene, identification of the gene in a blood sample is achieved. The hybridization probe may be detecting the gene at the DNA level or the RNA level.

As used herein, the phrase “increased risk of pre-term birth” refers to a situation wherein the risk of giving birth before 37 weeks gestational age is increased relative to a normal healthy pregnancy. For example, an increased risk of pre-term birth is where the risk of giving birth before 37 weeks gestational age is more than 1%, such as more than 10%.

As used herein, the term “ligand” refers to a compound (such as a small molecule, a protein, a nucleic acid, an aptamer, a peptide, a carbohydrate, etc.) having affinity towards a given amnion and/or chorion cell marker. The ligand, e.g., an antibody, may comprise a detectable marker, such as a reporter dye, fluorophore, or enzyme. Upon binding of the ligand to the cell marker, identification of the cell marker in a blood sample is achieved.

As used herein, the term “log 2 fold difference” refers to the difference in the expression of a given amnion and/or chorion cell marker between two samples, on a log 2 scale. For example, if the amount in a first sample is the double of the amount in a second sample, the log 2 fold difference is 1. Similarly, a log 2 fold difference of 10 means that the amount of cell marker in a first sample is 1024 times the amount in a second sample.

In one embodiment, a log 2 fold difference between the amount of a given amnion and/or chorion cell marker in a blood sample or fraction thereof isolated from a pregnant woman and the amount in a control of at least 5 is indicative of pre-term birth or an associated clinical condition or an increased risk of pre-term birth or an associated clinical condition of the pregnant woman.

In one embodiment, a log 2 fold difference between the amount of a given amnion and/or chorion cell marker in a blood sample or fraction thereof isolated from a pregnant woman and the amount in a control of at least 8 is indicative of pre-term birth or an associated clinical condition or an increased risk of pre-term birth or an associated clinical condition of the pregnant woman.

In one embodiment, a log 2 fold difference between the amount of a given amnion and/or chorion cell marker in a blood sample or fraction thereof isolated from a pregnant woman and the amount in a control of at least 10 is indicative of pre-term birth or an associated clinical condition or an increased risk of pre-term birth or an associated clinical condition of the pregnant woman.

In one embodiment, a log 2 fold difference between the amount of a given amnion and/or chorion cell marker in a blood sample or fraction thereof isolated from a pregnant woman and the amount in a control of at least 11 is indicative of pre-term birth or an associated clinical condition or an increased risk of pre-term birth or an associated clinical condition of the pregnant woman.

In one embodiment, a log 2 fold difference between the amount of a given amnion and/or chorion cell marker in a blood sample or fraction thereof isolated from a pregnant woman and the amount in a control of at least 12 is indicative of pre-term birth or an associated clinical condition or an increased risk of pre-term birth or an associated clinical condition of the pregnant woman.

In one embodiment, a log 2 fold difference between the amount of a given amnion and/or chorion cell marker in a blood sample or fraction thereof isolated from a pregnant woman and the amount in a control of at least 15 is indicative of pre-term birth or an associated clinical condition or an increased risk of pre-term birth or an associated clinical condition of the pregnant woman.

As used herein, the phrase “naturally occurring antibody” refers to heterotetrameric glycoproteins capable of recognizing and binding an antigen and comprising two identical heavy (H) chains and two identical light (L) chains inter-connected by disulfide bonds. Each heavy chain comprises a heavy chain variable region (abbreviated herein as VH) and a heavy chain constant region (abbreviated herein as CH). Each light chain comprises a light chain variable region (abbreviated herein as VL) and a light chain constant region (abbreviated herein as CL). The VH and VL regions can be further subdivided into regions of hypervariability, termed complementarity determining regions (CDRs), interspersed with regions that are more conserved, termed framework regions (FRs). Antibodies may comprise several identical heterotetramers. Antibodies may also be generated using immunization of suitable animals such as mice, rat, goat, rabbit, horse etc.

As used herein, the term “pre-term birth” refers to a premature birth, which generally refers to the birth of a baby before 37 weeks gestational age.

Amnion and/or Chorion Cell Markers.

In one embodiment, the presence of amnion and/or chorion cells is determined by detecting one or more specific amnion and/or chorion cell markers. The specific amnion and/or chorion cell markers may be differentially expressed in amnion and/or chorion cells compared to a blood sample or a fraction thereof isolated from a pregnant woman.

In one embodiment, a method of determining pre-term birth or an associated clinical condition or an increased risk of pre-term birth or an associated clinical condition is provided, the method comprising the steps of: (a) providing a blood sample or a fraction thereof isolated from a pregnant woman; (b) contacting the sample with i) a ligand directed to an amnion and/or chorion cell marker or ii) a hybridization probe comprising at least 10 contiguous nucleotides complementary to a gene encoding the amnion and/or chorion cell marker; and (c) detecting the amnion and/or chorion cell marker in the blood sample or fraction thereof of a); wherein the presence of an amnion and/or chorion cell marker is indicative of pre-term birth or an associated clinical condition or an increased risk of pre-term birth or an associated clinical condition of the pregnant woman.

In one embodiment, a method of determining pre-term birth or an associated clinical condition or an increased risk of pre-term birth or an associated clinical condition is provided, the method comprising the steps of: (a) contacting a sample from a pregnant woman or a fraction thereof with i) a ligand directed to an amnion and/or chorion cell marker or ii) a hybridization probe comprising at least 10 contiguous nucleotides complementary to a gene encoding the amnion and/or chorion cell marker; and (c) detecting the amnion and/or chorion cell marker in the blood sample or fraction thereof of a); wherein the presence of an amnion and/or chorion cell marker is indicative of pre-term birth or an associated clinical condition or an increased risk of pre-term birth or an associated clinical condition of the pregnant woman.

In one embodiment, the method further comprises: (a) determining the amount of amnion and/or chorion cell marker in the blood sample or fraction thereof; and (b) comparing the amount of amnion and/or chorion cell marker to a control, wherein an amount of amnion and/or chorion cell marker in the blood sample or fraction thereof higher than the amount in a control is indicative of pre-term birth or an associated clinical condition or an increased risk of pre-term birth or an associated clinical condition of the pregnant woman.

The amount of amnion and/or chorion cell marker in a control may be a pre-determined value. In one embodiment, the amount of amnion and/or chorion cell marker in a control is the amount of amnion and/or chorion cell marker in the blood of a pregnant woman not at risk of pre-term birth. In one embodiment, the amount of amnion and/or chorion cell marker in a control is a pre-determined value based on an average amount of amnion and/or chorion cell marker in the blood of a group of pregnant women not at risk of pre-term birth.

In one embodiment, the method comprises detection of the one or more amnion and/or chorion cell markers at the protein level, (the DNA level) and/or the RNA level.

Detection of Markers

In one embodiment, the method further comprises detecting an epithelial marker, such as a marker selected from the group consisting of CK1, CK2, CK3, CK4, CK5, CK6, CK7, CK8, CK9, CK10, CK12, CK13, CK14, CK15, CK16, CK17, CK18, CK19. The skilled person knows that “CK” and “KRT” are used interchangeably for the same proteins. As such, in a preferred embodiment, the epithelial marker is a KRT selected from table 1 and/or table 2.

In another embodiment, the method further comprises detecting a maternal marker, such as a marker selected from the group consisting of CD14 and CD45. A maternal marker can be used for negative selection of cells originating from the mother, hematopoietic markers present on the white blood cells are especially suited for such a purpose. In another embodiment, the method further comprises detecting a mesenchymal marker, such as Vimentin.

Samples. In one embodiment, the blood sample from a pregnant woman is whole blood, i.e. the blood has not been subjected to any fractionations before determining the presence of amnion and/or chorion cells. It is desirable to obtain as large a maternal blood sample as possible in order to increase the total number of potential amnion and/or chorion cells. Accordingly, the size of the maternal blood sample of step a in the method described in the first embodiment is preferably in the range of 0.5 to 50 ml, such as in the range of 1 to 40 ml, such as from 5 to 35 ml or 10 to 30 ml.

The provided blood sample is preferably isolated from a pregnant woman after 20+0 weeks of gestation. Thus, the blood sample is preferable isolated from a pregnant woman after 20, such as after 21+0, such as after 22+0, such as after 23+0, such as after 24+0, such as after 25+0, such as after 26+0, such as after 27+0, such as after 28+0, such as after 29+0, such as after 30+0, such as after 31+0, such as after 32+0, such as after 33+0, such as after 34+0, such as after 35+0, such as after 36+0 weeks of gestation. In a preferred embodiment, the blood sample is isolated between 20+0 and 37+6 weeks of gestation, such as between or 24+0 and 37+6 weeks of gestation.

Also, according to the invention the sample may be diluted or concentrated at any time during the method. The sample may be diluted at least 1.5 times, such as twice, more preferred at least three times, such as five times by adding isotonic buffers, such as saline solutions, phosphate buffered saline solutions, PBS, and/or suitable growth media, such as basal media, and tissues growth media. A method step may include dilution of a sample by addition of various components allocated for the specific method step.

For carrying out the method different method steps may be advantageous, such as, concentrating the sample, e.g., to reduce the volume without removing any cells. The sample volume may be decreased to less than 80%, such as 70, or 60 or 50% of the original sample volume, or even preferable to less than 40%, such as 25% of the original sample volume. A concentration step may be centrifugation. The method may according to the invention comprise one or more concentration steps. Centrifugation is a preferred method for concentrating the cells. In order to avoid damages of cells a mild centrifugation is preferred, such as 50-700×G for 10 minutes. In one embodiment, the blood sample is isolated from the pregnant woman after 20 weeks of gestation.

Detection and/or Quantification.

In one embodiment, the presence of amnion and/or chorion cells is determined by detection of one or more specific amnion and/or chorion cell markers. In another embodiment, the one or more amnion and/or chorion cell markers are detected at the protein level. This may be accomplished by contacting the blood sample or a fraction thereof isolated from a pregnant woman with a ligand directed to the amnion and/or chorion cell markers. In another embodiment, the one or more amnion and/or chorion cell markers are detected at the RNA level. This may be accomplished by contacting the blood sample or a fraction thereof isolated from a pregnant woman with a hybridization probe comprising nucleotides complementary to a gene encoding the amnion and/or chorion cell markers.

The ligand directed to an amnion and/or chorion cell marker and/or the hybridization probe comprising nucleotides complementary to a gene encoding the amnion and/or chorion cell marker may comprise a reporter dye, thereby allowing detection of and/or quantification of the amnion and/or chorion cell marker protein, or RNA in a blood sample.

The presence of a given amnion and/or chorion cell marker in a blood sample or a fraction thereof isolated from a pregnant woman may be determined by the detection of a signal from the ligand directed to the amnion and/or chorion cell marker and/or the hybridization probe comprising nucleotides complementary to a gene encoding the amnion and/or chorion cell marker upon contacting the ligand and/or hybridization probe to a blood sample or a fraction thereof isolated from a pregnant woman.

Thus, one embodiment comprises detecting the presence of the ligand or the hybridization probe in a blood sample or a fraction thereof from a pregnant woman.

Detection may be enabled by labeling the ligand or the hybridization probe with a reporter dye, such as a fluorescent dye or other dyes suitable for detection. Thus, the method may e.g. be fluorescent in-situ hybridization (FISH). The probe may comprise a quencher as well as a fluorophore or a FRET pair as described below, which enables detection of hybridization probes bound to their target sequences. Alternatively, or additionally, probes binding to their targets are separated from non-binding probes by one or more washing steps.

Identification may also be done using immunostaining using a ligand such as an antibody. Identification may also be done using multicolor FISH, multicolor immunostaining, Single-Nucleotide Polymorphism (SNP), simple sequence repeats (SSR), or short tandem repeats (STR). For example, different hybridization probes with different fluorescent labels may be used simultaneously or two (or more) different antibodies with different fluorescent labels may be used simultaneously.

The amount of a given amnion and/or chorion cell marker in a blood sample or a fraction thereof isolated from a pregnant woman may be determined by quantifying the signal obtained from the ligand directed to the amnion and/or chorion cell marker and/or the hybridization probe comprising nucleotides complementary to a gene encoding the amnion and/or chorion cell marker upon contacting the ligand and/or hybridization probe to a blood sample or a fraction thereof isolated from a pregnant woman.

The difference in the amount of a given amnion and/or chorion cell marker in a blood sample or a fraction thereof isolated from a pregnant woman and the amount of the amnion and/or chorion cell marker in a control may be presented as a log 2 fold difference.

Amnion and/or Chorion Cell Markers

The present invention is based on the finding that specific amnion and/or chorion cells are present and/or present in an increased amount in the blood of pregnant women who are about to or at increased risk of giving birth prematurely. The amnion and/or chorion cells may therefore be used as biomarkers for determining pre-term birth or an associated clinical condition or an increased risk of pre-term birth or an associated clinical condition from a blood sample of a pregnant woman. The presence of amnion and/or chorion cells may be determined by detection of specific amnion and/or chorion cell markers.

A list of amnion and/or chorion cell markers that may be used for determining the presence and/or amount of amnion and/or chorion cells in the blood of a pregnant women who are at increased risk of giving birth prematurely are summarized below in table 1.

TABLE 1
Amnion and/or chorion cell markers that may be used for determining the presence
and/or amount of amnion and/or chorion cells in the blood of a pregnant woman.

| AMNION | | | CHORION | | |
|---|---|---|---|---|---|
| | | LOG2 FOLD | | | LOG2 FOLD |
| MARKER | ACCESSION NR. | CHANGE | MARKER | ACCESSION NR. | CHANGE |
| IGF2^{#∞} | NM_000612 | −19.4705 | IGF2^{#∞} | NM_000612 | −15.6259 |
| MUC16^{#} | NM_024690 | −13.4421 | THY1^{#} | NM_001311160 | −14.0688 |
| KRT5 | NM_000424 | −11.6926 | DCN | NM_001920 | −12.8043 |
| TSPAN1 | NM_005727 | −11.635 | DIO2^{#} | NM_000793 | −11.9493 |
| IGFBP3^{∞} | NM_000598 | −11.3469 | IGFBP3^{∞} | NM_000598 | −11.829 |
| SERPINB10^{∞} | NM_005024 | −11.3159 | IGFBP2 | NM_000597 | −11.6142 |
| UPK1B^{#} | NM_006952 | −11.2787 | SPARCL1 | NM_001128310 | −11.1234 |
| CADPS2 | NM_001009571 | −11.2385 | NNMT | NM_006169 | −10.943 |
| LAMC2 | NM_005562 | −11.1718 | LPHN3^{#} | NM_001322246 | −10.9382 |
| AHNAK2 | NM_001350929 | −11.06 | CRYAB^{∞} | NM_001289807 | −10.8649 |
| EMP1^{#} | NM_001423 | −11.0187 | PEG3 | NM_001146184 | −10.2634 |
| FN1 | NM_001306129 | −10.8042 | FLT1^{#} | NM_001159920 | −10.1574 |
| FBN1^{∞} | NM_000138 | −10.6945 | GPX8^{#∞} | NM_001008397 | −9.90866 |
| MET | NM_000245 | −10.6592 | FBN1^{∞} | NM_000138 | −9.82739 |
| PVRL4^{#} | NM_030916 | −10.5813 | NPR3^{#} | NM_000908 | −9.80858 |
| A2ML1 | NM_001282424 | −10.4185 | AOC1 | NM_001091 | −9.79635 |
| DSP | NM_001008844 | −10.2635 | ITGB8 | NM_002214 | −9.65012 |
| THSD4 | NM_001286429 | −10.1623 | RXFP1^{#} | NM_001253727 | −9.52972 |
| CRYAB^{∞} | NM_001289807 | −10.1597 | SPOCK1 | NM_004598 | −9.5196 |
| KRT17 | NM_000422 | −10.0906 | CYP11A1 | NM_000781 | −9.48987 |
| KRT18^{∞} | NM_000224 | −10.09 | COL4A2 | NM_001846 | −9.42377 |
| PDLIM4 | NM_001131027 | −10.0882 | KRT18^{∞} | NM_000224 | −9.4108 |
| COL17A1 | NM_000494 | −10.049 | CNR1^{#} | NM_001160226 | −9.39036 |
| PRTG^{#∞} | NM_173814 | −9.80969 | SEMA3A | NM_006080 | −9.38343 |
| DKK3 | NM_001018057 | −9.8003 | SERPINE1 | NM_000602 | −9.37988 |
| PLS3 | NM_001136025 | −9.7741 | IL1R1 | NM_000877 | −9.3607 |
| COL1A2^{∞} | NM_000089 | −9.67879 | FBLN1 | NM_001996 | −9.3031 |
| GPX8^{#∞} | NM_001008397 | −9.58492 | COL1A2^{∞} | NM_000089 | −9.29572 |
| DPYSL3 | NM_001197294 | −9.53715 | UCHL1 | NM_004181 | −9.25047 |
| TPPP3 | NM_015964 | −9.53616 | RAI2 | NM_001172732 | −9.24456 |
| SHROOM3 | NM_020859 | −9.44555 | TGM2 | NM_001323316 | −9.2418 |
| | | | PRLR^{#} | NM_000949 | −9.18432 |
| | | | FSTL3 | NM_005860 | −9.10492 |
| | | | SERPINB10^{∞} | NM_005024 | −9.09163 |
| | | | BCAR1 | NM_001170714 | −9.0538 |
| | | | THSD4 | NM_001286429 | −9.0091 |
| | | | PRTG^{#∞} | NM_173814 | −8.95802 |
| | | | FERMT2 | NM_001134999 | −8.94575 |
| | | | PKP2 | NM_001005242 | −8.8545 |
| | | | P4HA2 | NM_001017973 | −8.83204 |
| | | | TEAD1 | NM_021961 | −8.82638 |
| | | | AQPEP | NM_173800 | −8.77963 |

^{#}Markers expressed on cell membrane
^{∞}Common markers between Amnion and Chorion

TABLE 2
Further markers that can be used in methods of the invention
ADDITIONAL MARKERS

| | |
|---|---|
| Extra Cytokeratins | |
| KRT7 | NM_005556 |
| KRT8 | NM_001256293 |
| KRT19 | NM_002276 |
| KRT12 | NM_000223 |
| Vimentin | |
| VIM | NM_003380 |
| Hematopoietic | |
| markers | |
| CD14 | NM_000591 |
| CD45 | NM_080921 |

In one embodiment, the one or more amnion and/or chorion cell markers are selected from the group consisting of: IGF2, IGFPB3, SERPINB10, FBN1, CRYAB, KRT18, PRTG, COL1A2, GPX8, MUC16, KRT5, TSPAN1, UPK1B, CADPS2, LAMC2, AHNAK2, EMP1, FN1, MET, PVRL4, A2ML1, DSP, THSD4, KRT17, PDLIM4, COL17A1, DKK3, PLS3, DPYSL3, TPPP3, SHROOM3, THY1, DCN, DIO2, IGFBP2, SPARCL1, NNMT, LPHN3, PEGS, FLT1, NPR3, AOC1, ITGB8, RXFP1, SPOCK1, CYP11A1, COL4A2, CNR1, SEMA3A, SERPINE1, IL1R1, FBLN1, UCHL1, RAI2, TGM2, PRLR, FSTL3, BCAR1, THSD4, FERMT2, PKP2, P4HA2, TEAD1, or AQPEP.

In one embodiment, the one or more amnion and/or chorion cell markers are selected from the group consisting of IGF2, MUC16, THY1, DIO2, UPK1B, LPHN3, EMP1, FLT1, GPX8, PVRL4, NPR3, RXFP1, CNR1, PRTG, PRLR, PRTG. These markers are expressed on cell membrane and therefore are good candidates for enrichment of fetal membrane cells using technologies known for the person of skill in the art, such as magnetic activated cell sorting.

In one embodiment, the one or more amnion and/or chorion cell markers are elected among amnion cells. Preferred markers for amnion cells are MUC16, UPK1B, and EMP1.

In one embodiment, the one or more amnion and/or chorion cell markers are elected among chorion cells. Preferred markers for chorion cells are FLT1/VEGFR1, RXFP1, CNR1, and PRLR.

In one embodiment, the one or more amnion and/or chorion cell markers are selected among markers expressed on both chorion cells and amnion cells but not on maternal blood cells. Preferred such markers are IGF2, IGFBP3, SERPINB10, CRYAB, FBN1, GPX8, FBN1, KRT18, PRTG, or COL1A2. Particular preferred markers are expressed on the cell membrane, such as IGF2, GPX8, and PRTG.

In one embodiment, the one or more amnion and/or chorion cell markers originate from the amnion and/or the chorion. In one embodiment, the number of cell markers selected from the amnion and/or chorion cell markers are: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 49, 49, 50, 51, 52, 53, 54, 55, 56, 57, 58, 59, 60, 61, 62, 63 or all the markers in Table 1.

In one embodiment, 1, 2, 3, 4, 5, 6, 7, 8, 9, 10, 11, 12, 13, 14, 15, 16, 17, 18, 19, 20, or 21, amnion and/or chorion cell markers are selected from the group consisting of MUC16, UPK1B, EMP1, PVRL4, THY1, DIO2, LPHN3, FLT1, NPR3, RXFP1, CNR1, PRLR, IGF2, IGFBP3, SERPINB10, FBN1, CRYAB, KRT18, PRTG, COL1A2 and GPX8. In one embodiment, the amnion and/or chorion cell markers are selected in order.

In one embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 amnion and/or chorion cell markers originate from the amnion. Thus, in one embodiment, the one or more amnion and/or chorion cell markers are selected from the group consisting of: IGF2, MUC16, KRT5, TSPAN1, IGFBP3, SERPINB10, UPK1B, CADPS2, LAMC2, AHNAK2, EMP1, FN1, FBN1, MET, PVRL4, A2ML1, DSP, THSD4, CRYAB, KRT17, KRT18, PDLIM4, COL17A1, PRTG, DKK3, PLS3, COL1A2, GPX8, DPYSL3, TPPP3 and SHROOM3. In one embodiment, the amnion and/or chorion cell markers are selected in order.

In one embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 amnion and/or chorion cell markers originate from the chorion. Thus in one embodiment, the one or more amnion and/or chorion cell markers are selected from the group consisting of: IGF2, THY1, DCN, DIO2, IGFBP3, IGFBP2, SPARCL1, NNMT, LPHN3, CRYAB, PEG3, FLT1, GPX8, FBN1, NPR3, AOC1, ITGB8, RXFP1, SPOCK1, CYP11A1, COL4A2, KRT18, CNR1, SEMA3A, SERPINE1, IL1R1, FBLN1, COL1A2, UCHL1, RAI2, TGM2, PRLR, FSTL3, SERPINB10, BCAR1, THSD4, PRTG, FERMT2, PKP2, P4HA2, TEAD1 and AQPEP. In one embodiment, the amnion and/or chorion cell markers are selected in order.

In one embodiment, 1, 2, 3, 4, 5, 6, 7, 8, or 9 amnion and/or chorion cell markers originate from the chorion and from the amnion. Thus, in one embodiment, the one or more amnion and/or chorion cell markers are selected from the group consisting of: IGF2, IGFBP3, SERPINB10, FBN1, CRYAB, KRT18, PRTG, COL1A2 and GPX8. In one embodiment, the amnion and/or chorion cell markers are selected in order.

In one embodiment, 1, 2, 3, 4, 5, 6, 7 or 8 amnion and/or chorion cell markers originate from the chorion and from the amnion. Thus, in one embodiment, the one or more amnion and/or chorion cell markers are selected from the group consisting of: MUC16, UPK1B, EMP1, GPX8, FLT1/VEGFR1, RXFP1, CNR1 and PRLR. In one embodiment, the amnion and/or chorion cell markers are selected in order.

In one embodiment, 1, 2, 3, 4, 5, 6, 7, 8, 9, 10, 11, 12, 13, 14 or 15 amnion and/or chorion cell markers are present in the cell membrane. Thus, in one embodiment, the one or more amnion and/or chorion cell markers are selected from the group consisting of: IGF2, MUC16, UPK1B, EMP1, PVRL4, THY1, DIO2, LPHN3, FLT1, NPR3, RXFP1, CNR1, PRLR, PRTG and GPX8. At least one of the advantages of using a marker present in the cell membrane, is that the marker may have an extracellular epitope that is accessible from the extracellular side. In one embodiment, the amnion and/or chorion cell markers are selected in order.

In one embodiment, 1, 2, 3, 4, 5, 6 or 7, amnion and/or chorion cell markers originate from the amnion and is present in the cell membrane. Thus, in one embodiment, the one or more amnion and/or chorion cell markers are selected from the group consisting of: IGF2, MUC16, UPK1B, EMP1, PVRL4, PRTG and GPX8. In one embodiment, the amnion and/or chorion cell markers are selected in order.

In one embodiment, 1, 2, 3, 4, 5, 6, 7, 8, 9, 10 or 11 amnion and/or chorion cell markers originate from the chorion and is present in the cell membrane. Thus, in one embodiment, the one or more amnion and/or chorion cell markers are selected from the group consisting of: IGF2, THY1, DIO2, LPHN3, FLT1, NPR3, RXFP1, CNR1, PRLR, PRTG and GPX8. In one embodiment, the amnion and/or chorion cell markers are selected in order.

In one embodiment, 1, 2, 3, 4 or 5 amnion and/or chorion cell markers are intracellularly located. Thus, in one embodiment, the one or more amnion and/or chorion cell markers are selected from the group consisting of: UCHL1, AHNAK2, PDLIM3, DPYSL3 and PDLIM4. In one embodiment, the amnion and/or chorion cell markers are selected in order.

In one embodiment, 1 amnion and/or chorion cell marker originate from the amnion and are intracellularly located. Thus, in one embodiment, the one or more amnion and/or chorion cell markers is UCHL1.

In one embodiment, 1, 2, 3 or 4 amnion and/or chorion cell markers are intracellularly located and originate from the chorion. Thus, in one embodiment, the one or more amnion and/or chorion cell markers are selected from the group consisting of: AHNAK2, PDLIM3, DPYSL3 and PDLIM4.

In one embodiment, the difference between the expression of the amnion and/or chorion cell markers in amnion and/or chorion cells compared with the expression in a blood sample or fraction thereof isolated from a pregnant woman may be represented as a log 2 fold difference. In one embodiment, a marker is chosen based on the log 2 fold difference. In a further embodiment, the marker selected based on the log 2 fold difference is further selected based on being present in the cell membrane. When a suitable marker is selected, an antibody capable of binding to an extracellular epitope may be selected.

In one embodiment, the log 2 fold difference between the expression of the amnion and/or chorion cell markers in amnion and/or chorion cells compared with the expression in a blood sample or fraction thereof isolated from a pregnant woman is at least 15. Thus, in one embodiment, the amnion and/or chorion cell marker is IGF2.

In one embodiment, the log 2 fold difference between the expression of the amnion and/or chorion cell markers in amnion and/or chorion cells compared with the expression in a blood sample or fraction thereof isolated from a pregnant woman is at least 12. Thus, in one embodiment, the amnion and/or chorion cell marker is selected from the group consisting of: IGF2, MUC16, THY1 and DCN.

In one embodiment, the amnion and/or chorion cell marker originates from the amnion and the log 2 fold difference between the expression of the amnion and/or chorion cell markers in amnion and/or chorion cells compared with the expression in a blood sample or fraction thereof isolated from a pregnant woman is at least 12. Thus, in one embodiment, the amnion and/or chorion cell marker is selected from the group consisting of: IGF2 and MUC16.

In one embodiment, the amnion and/or chorion cell marker originates from the chorion and the log 2 fold difference between the expression of the amnion and/or chorion cell markers in amnion and/or chorion cells compared with the expression in a blood sample or fraction thereof isolated from a pregnant woman is at least 12. Thus, in one embodiment, the amnion and/or chorion cell marker is selected from the group consisting of: IGF2, THY1 and DCN.

In one embodiment, the log 2 fold difference between the expression of the amnion and/or chorion cell markers in amnion and/or chorion cells compared with the expression in a blood sample or fraction thereof isolated from a pregnant woman is at least 11. Thus, in one embodiment, the amnion and/or chorion cell marker is selected from the group consisting of: IGF2, MUC16, THY1, DCN, KRT5, TSPAN1, IGFBP3, SERPINB10, UPK1B, CADPS2, LAMC2, AHNAK2, EMP1, DIO2, IGFBP2 and SPARCL1.

In one embodiment, the amnion and/or chorion cell marker originates from the amnion and the log 2 fold difference between the expression of the amnion and/or chorion cell markers in amnion and/or chorion cells compared with the expression in a blood sample or fraction thereof isolated from a pregnant woman is at least 11. Thus, in one embodiment, the amnion and/or chorion cell marker is selected from the group consisting of: IGF2, MUC16, KRT5, TSPAN1, IGFBP3, SERPINB10, UPK1B, CADPS2, LAMC2, AHNAK2 and EMP1.

In one embodiment, the amnion and/or chorion cell marker originates from the chorion and the log 2 fold difference between the expression of the amnion and/or chorion cell markers in amnion and/or chorion cells compared with the expression in a blood sample or fraction thereof isolated from a pregnant woman is at least 11. Thus, in one embodiment, the amnion and/or chorion cell marker is selected from the group consisting of: IGF2, THY1, DCN, DIO2, IGFBP3, IGFBP2 and SPARCL1.

In one embodiment, the log 2 fold difference between the expression of the amnion and/or chorion cell markers in amnion and/or chorion cells compared with the expression in a blood sample or fraction thereof isolated from a pregnant woman is at least 10. Thus, in one embodiment, the amnion and/or chorion cell marker is selected from the group consisting of: IGF2, MUC16, THY1, DCN, KRT5, TSPAN1, IGFBP3, SERPINB10, UPK1B, CADPS2, LAMC2, AHNAK2, EMP1, DIO2, IGFBP2, SPARCL1, FN1, FBN1, MET, PVRL4, A2ML1, DSP, THSD4, CRYAB, KRT17, KRT18, PDLIM4, COL17A1, NNMT, LPHN3, PEG3 and FLT1.

In one embodiment, the amnion and/or chorion cell marker originates from the amnion and the log 2 fold difference between the expression of the amnion and/or chorion cell markers in amnion and/or chorion cells compared with the expression in a blood sample or fraction thereof isolated from a pregnant woman is at least 10. Thus, in one embodiment, the amnion and/or chorion cell marker is selected from the group consisting of: IGF2, MUC16, KRT5, TSPAN1, IGFBP3, SERPINB10, UPK1B, CADPS2, LAMC2, AHNAK2, EMP1, FN1, FBN1, MET, PVRL4, A2ML1, DSP, THSD4, CRYAB, KRT17, KRT18, PDLIM4 and COL17A1.

In one embodiment, the amnion and/or chorion cell marker originates from the chorion and the log 2 fold difference between the expression of the amnion and/or chorion cell markers in amnion and/or chorion cells compared with the expression in a blood sample or fraction thereof isolated from a pregnant woman is at least 10. Thus, in one embodiment, the amnion and/or chorion cell marker is selected from the group consisting of: IGF2, THY1, DCN, DIO2, IGFBP3, IGFBP2, SPARCL1, NNMT, LPHN3, CRYAB, PEG3 and FLT1.

Ligand Directed to an Amnion and/or Chorion Cell Marker.

The presence and/or the amount of a given amnion and/or chorion cell may be determined by the detection of one or more specific amnion and/or chorion cell markers which may be detected and/or quantified by treatment of a blood sample or fraction thereof isolated from a pregnant woman with a ligand directed to the one or more amnion and/or chorion cell markers.

The use of a ligand for detecting and/or quantifying a given amnion and/or chorion cell marker allow for detection of the amnion and/or chorion cell marker at the protein level and/or the RNA level.

The ligand as used in the method of the invention is often an antibody, a peptide or an aptamer. A ligand as used in the method of the invention binds primarily to the cell marker(s) of interest, preferably with a higher affinity than binding to other cell markers. Thus, preferably the ligand binds primarily to the amnion and/or chorion cell markers.

The ligand may be an aptamer. Aptamers are nucleic acid based high-affinity ligands that bind to antigens such as proteins. They are typically identified using in vitro evolution techniques such as SELEX (systematic evolution of ligands by exponential enrichment). In SELEX, iterated rounds of selection and amplification of nucleic acids from an initial library is used for identification of high-affinity aptamers. Since the initial library is very large (e.g., 10¹⁴ different sequences) and sequences may be mutated during iterated rounds, identification of high affinity aptamers can now be done on a routine basis and such methods are known to the skilled artisan.

Often, aptamers are less than 50 nucleotides in length.

High affinity peptides may be generated using phage display. In phage display, a library of phages displaying peptides are selected against the target and subsequently amplified in an evolution process similar to SELEX. Various systems for phage display exist and the size of the peptide may be chosen to suit particular needs. In one embodiment, the peptides to be used with the method of the invention have a size of less than 50 amino acids.

Often the library is displayed as a scaffold, e.g., an antibody scaffold. Thus, phage display may be used to identify high affinity antibodies. Other in vitro evolution techniques for antibody generation involve mRNA display, ribosome display and covalent DNA display.

The ligand may also be an antibody. An antibody according to the invention is a polypeptide or protein capable of recognizing and binding an antigen comprising at least one antigen binding site. The antigen binding site preferably comprises at least one complementarity determining region (CDR). The antibody may be a naturally occurring antibody, a fragment of a naturally occurring antibody or a synthetic antibody.

Naturally occurring antibodies can include heterotetrameric glycoproteins capable of recognizing and binding an antigen and comprising two identical heavy (H) chains and two identical light (L) chains inter-connected by disulfide bonds. Each heavy chain comprises a heavy chain variable region (abbreviated herein as VH) and a heavy chain constant region (abbreviated herein as CH). Each light chain comprises a light chain variable region (abbreviated herein as VL) and a light chain constant region (abbreviated herein as CL). The VH and VL regions can be further subdivided into regions of hypervariability, termed complementarity determining regions (CDRs), interspersed with regions that are more conserved, termed framework regions (FRs). Antibodies may comprise several identical heterotetramers. Antibodies may also be generated using immunization of suitable animals such as mouse, rat, goat, rabbit, horse etc.

Antibodies used for the present invention may be either monoclonal or polyclonal. Methods of generating both types of antibodies are well known to the skilled artisan. In addition to in vitro evolution methods outlined above, monoclonal antibodies are typically prepared using hybridoma technology.

Hybridization Probe.

The presence and/or the amount of a given amnion and/or chorion cell marker may be detected and/or quantified by treatment of a blood sample or fraction thereof isolated from a pregnant woman with a hybridization probe comprising nucleotides complementary to a gene encoding the amnion and/or chorion cell marker.

Hybridization probes are nucleic acids having a sequence capable of hybridizing with a gene encoding a given amnion and/or chorion cell marker. The hybridization probe comprises nucleotides complementary to the gene. The hybridization probe may be labelled with a reporter dye. Upon hybridization of the hybridization probe with the gene, identification of the gene in a blood sample is achieved. The hybridization probe may be detecting the gene at the RNA level.

Hybridization probes may be used as generally in the art and are typically DNA or RNA, preferably DNA. In preferred embodiments, the probes are modified with non-natural nucleotides that improve binding affinity and/or binding specificity. Preferred examples of such non-natural nucleotides are LNA (locked nucleic acids), TINA (twisted intercalating nucleic acids), PNA (peptide nucleic acid), INA (intercalating nucleic acids), morpholino and 2′O-substituted RNA monomers such as 2′O-methyl RNA monomers and 2′O-(2-methoxyethyl) RNA.

The length of the probes may be any suitable length, such as in the range of 10 to 200 nucleotides, preferably between 10 and 30 nucleotides, more preferably 15-25 nucleotides and preferably, the probe is fully complementary to the gene encoding a given amnion and/or chorion cell marker.

In one embodiment the probe is at least 85% complementary to a gene encoding any of the proteins described in Table 1, such as at least 90% complementary, for example at least 95% complementary over the length of the probe. The probe may be complementary to the mRNA encoding the protein.

In one embodiment, the hybridization probe comprises at least 10 contiguous nucleotides complementary to a gene encoding the amnion and/or chorion cell marker. In one embodiment, the hybridization probe comprises at least 15 contiguous nucleotides complementary to a gene encoding the amnion and/or chorion cell marker. In one embodiment, the hybridization probe comprises at least 20 contiguous nucleotides complementary to a gene encoding the amnion and/or chorion cell marker.

Reporter Dye.

The hybridization probes and ligands to be used according to the invention may comprise or preferably be linked to a reporter dye. The hybridization probes or ligand are preferably covalently linked to a reported dye. The reporter dye is preferably a fluorescent reporter dye. Preferably, the reporter dye is selected from the group consisting of FAM™, TET™, JOE™, VIC™, SYBR® Green, 6 FAM, HEX, TET, TAMRA, JOE, ROX, Fluorescein, Cy3, Cy5, Cy5.5, Texas Red, Rhodamine, Rhodamine Green, Rhodamine Red, 6-CarboxyRhodamine 6G, Alexa Fluor, Oregon Green 488, Oregon Green 500, and/or Oregon Green 514.

In one embodiment, the hybridization probes also comprise a quenching dye. In a preferred embodiment, the quenching dye is selected from the group consisting of TAMRA™, Black Hole Quencher™, DABCYL, BHQ-1, BHQ-2, DDQ I, DDQ II, and/or Eclipse Dark Quencher. The use of reporter and quenching dye is desirable because it allows various kinds of quantifications in addition to identification.

Typically, the reporter dye and the quencher dye are located near each other in the hybridization probe, allowing light- or laser-induced fluorescence emitted by the reporter to be quenched by the quencher dye. When the oligonucleotide binds to a complementary template strand, the reporter dye and the quencher dye are separated from each other such that the quencher no longer quenches the signal from the reporter, i.e. hybridization can be detected.

Thus, in one embodiment, the hybridization probe is capable of forming a stem-loop structure, wherein the quencher and reporter dye are brought into proximity in the stem. In one embodiment, the oligonucleotide is a so-called molecular beacon. The quencher and the reporter are no longer in proximity, when the molecular beacon base pairs to a template strand. Therefore the laser-induced signal from the reporter dye is no longer quenched.

Instead of using a reporter dye and a quencher dye, a so-called FRET (fluorescence resonance energy transfer) pair comprising a donor fluorophor and an acceptor fluorophor may be used. When the donor fluorophor is excited by an external light source, it emits light at a wavelength, which excites the acceptor fluorophor, which in turn emits light at a different wavelength, which can be detected and measured. The energy is only transferred from the donor to the acceptor if the donor fluorophor and acceptor fluorophor are in close proximity.

Examples of FRET pairs include: BFP-YFP, CFP-YFP, GFP-DsRed, GFP-Cy3, GFP-mOrange, YFP-RFP, FAM-ROX, FAM-Cy5, FAM-Hex, FAM-TAMRA, and/or Cy3-Cy5.

Detection and/or Quantification of Amnion and/or Chorion Cell Markers and Diagnosis.

Preferably, the methods of the invention may be used for predicting pre-term birth or an associated clinical condition or an increased risk of pre-term birth or an associated clinical condition from a blood sample from a pregnant woman.

Early prediction of pre-term birth and/or increased risk of pre-term birth is highly important in order to provide treatment of the pregnant woman to postpone the birth for as long as possible or to be prepared for providing extensive care of the infant born pre-maturely.

Thus, in one embodiment, treatment is provided to the pregnant woman identified as being about to give birth prematurely or being at increased risk of giving birth prematurely in order to minimize the risk of pre-term birth or ameliorating the consequences of pre-term birth, such as hospitalizing the individual.

Clinical Condition Associated with Pre-Term Birth or an Increased Risk of Pre-Term Birth.

Pre-term birth and increased risk of pre-term birth may be accompanied by associated clinical conditions. Identification of pregnant women about to give birth prematurely or at increased risk of pre-term birth is therefore highly important in order to treat such associated clinical conditions. In one embodiment, the clinical condition associated with pre-term birth is preeclampsia.

### Example 1

Flash-frozen tissues of the chorionic and amnionic membranes received from Dr. Ramkumar Menon's lab were subjected to RNA sequencing for the quantification of gene transcripts in order to find genes that were differentially expressed in the cells from these membranes as compared to maternal blood cells. Briefly, RNA was extracted using the RNeasy UCP kit (Qiagen), quantified on a Nanodrop, and checked for integrity on an Agilent Bioanalyzer. RNA sequencing was performed by Qiagen/Exiqon in Hilden, Germany using the Illumina TruSeq Stranded Total RNA Library Prep Kit. A list of differentially expressed genes was generated. The identified markers can be seen in table 1. There were 31 genes that were overexpressed in the amnion, of which 7 genes were expressed on the cell membrane. However, there were 42 genes that were overexpressed in the chorion, of which 11 were on the cell membrane. These 15 genes (GPX8, PRTG and IGF2 are common between amnion and chorion), expressed on the cell membrane are initial targets for use as biomarkers for the isolation of the amnion and chorion cells from maternal blood using ARCEDI technology.

Identification of fetal membrane cell markers. Fetal membrane cells collected from normal term (not in labor) deliveries were subjected to RNAseq analysis. Markers in these cells that were differentially expressed compared to maternal blood cells were used for enriching fetal membrane cells from maternal blood.

### Example 2

The present inventors determined whether shed amniochorion membrane cells can reach the maternal side either through tissue layers (microfractures) or through feto-maternal circulation, and these cells can be identified in maternal blood to serve as a marker for fetal membrane physiology and function.

Methods for enriching fetal membrane cells from maternal blood can include, e.g., those taught in U.S. Pat. No. 9,429,520, and International Patent Publication No. WO2012/062325, which are directed to relevant portions incorporated herein by reference. Briefly, the method can include fixation of cells in a maternal blood sample, which greatly increases the stability of fetal cells in a maternal blood sample, while allowing enrichment and identification of fetal cells.

Blood sampling. Peripheral blood samples of 30 mL were obtained from pregnant women at 36+ weeks of gestational age, and from 10-14 weeks of gestational age. All blood samples were collected in Streck tubes and processed within 4 hours after collection. The gender of the fetus was determined by real-time PCR of free fetal DNA using Y-chromosome-specific genes.

Whole blood fixation and red blood cell lysis. Blood samples were fixed within 15 minutes of drawing in 2% formaldehyde in PBS. After lysing red blood cells, nucleated cells were harvested using methods reported previously.

Enrichment of Fetal Membrane Cells by Magnetic-Activated Cell Sorting (MACS).

Enrichment of fetal membrane cells was performed using 9 different primary antibodies targeting 8 different markers in the fetal membrane cells, mixed together according to the manufacturer's basic protocols with slight modifications. Markers and antibodies against these markers were used as shown in example 3. The cell suspension was incubated with the antibodies for 30 minutes, washed twice with 14 mL Magnetic Activated Cell Sorting (MACS) buffer (4° C.), recovered by centrifugation, and resuspended in MACS buffer.

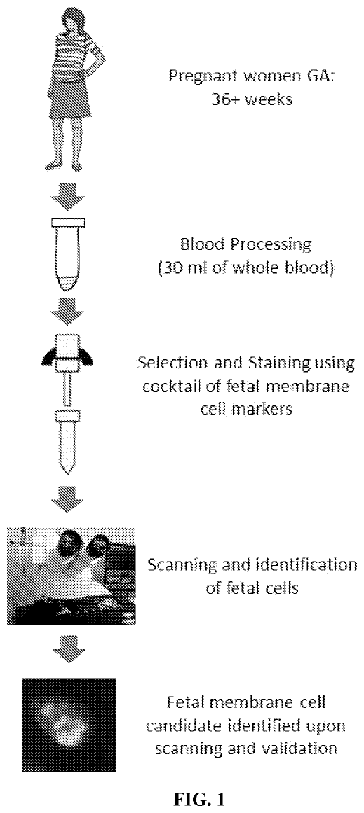

The approach of identifying fetal membrane cells from maternal blood samples is shown in FIG. 1. A blood sample is obtained from a pregnant woman. The whole blood sample (e.g., 30 ml) is processed to identify cells and cell fragments. The cells and cell fragments are then stained for the cell markers taught herein. Fetal cells are then scanned and identified. Finally, fetal membrane cell candidates are identified upon scanning and validation.

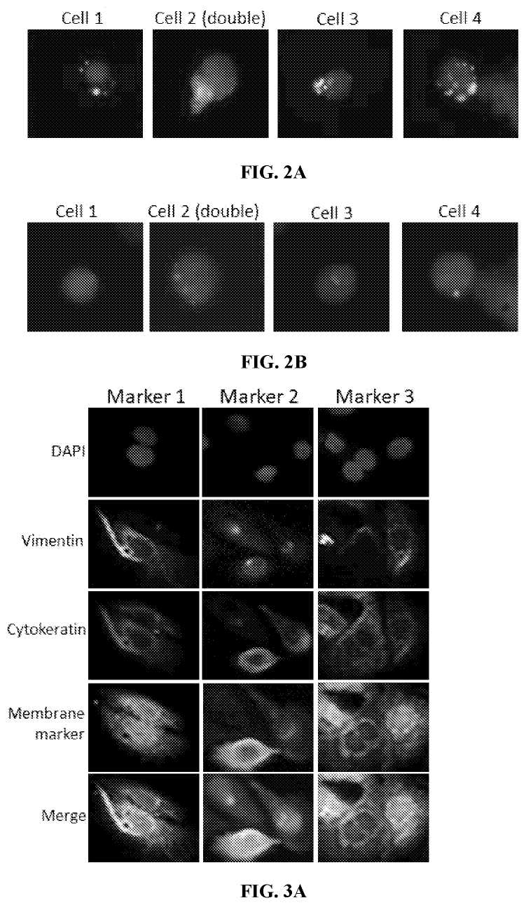

From three pregnant women at gestation age of 36+ thirty-six amnion/chorion cells were enriched by MACS and characterized by staining with anti-cytokeratin antibodies from 3 blood samples from pregnant women carrying male fetuses at term (FIG. 2A). Fetal sex was confirmed by FISH (FIG. 2B). FISH was carried out with X- and Y-chromosome specific probes. Results from the fetal membrane cell enrichment and identification are presented in table 3.

TABLE 3
fetal membrane cell identified in pregnant
woman at 36+ weeks of gestation.

| | Fetal | Fetal | | | Extra |
|---|---|---|---|---|---|
| | Membrane | Membrane | FMCCs | FMCCs | FMCs |
| | Candidate | Cells (FMCs) | with no | lost | found |
| Sample | Cells | confirmed | XY-FISH | during | after |
| No | (FMCCs) | by XY-FISH | signals^{#} | XY-FISH | Y-scan |
| PTB-1 | 7 | 5 | 2 | 0 | 3 |
| PTB-2 | 18 | 0 | 15 | 3 | —* |
| PTB-3 | 11 | 11 | 0 | 0 | —* |

^{#}XY-FISH failed on these cells.
*No Y-scan performed.

No amnion/chorion cells were identified from the enriched samples from two pregnant women at gestational age of 10-14 weeks (Table 4). Showing that these markers are only expressed in fetal membrane cells and not in other types of fetal cells (placental/trophoblast). The data showing no fetal membrane cells at 10-14 weeks after gestation means that these markers do not enrich other types of fetal cells (placental/trophoblasts), but only fetal membrane cells. Hence, these markers are good candidates to study the conditions related to term parturition and early parturition (pre term birth).

TABLE 4
fetal membrane cell enrichment from pregnant
woman at 10-14 weeks of gestation.

| | Membrane Candidate |
|---|---|
| Sample No | Cells (FMCCs) |
| 2529 | 0 |
| 2530 | 0 |

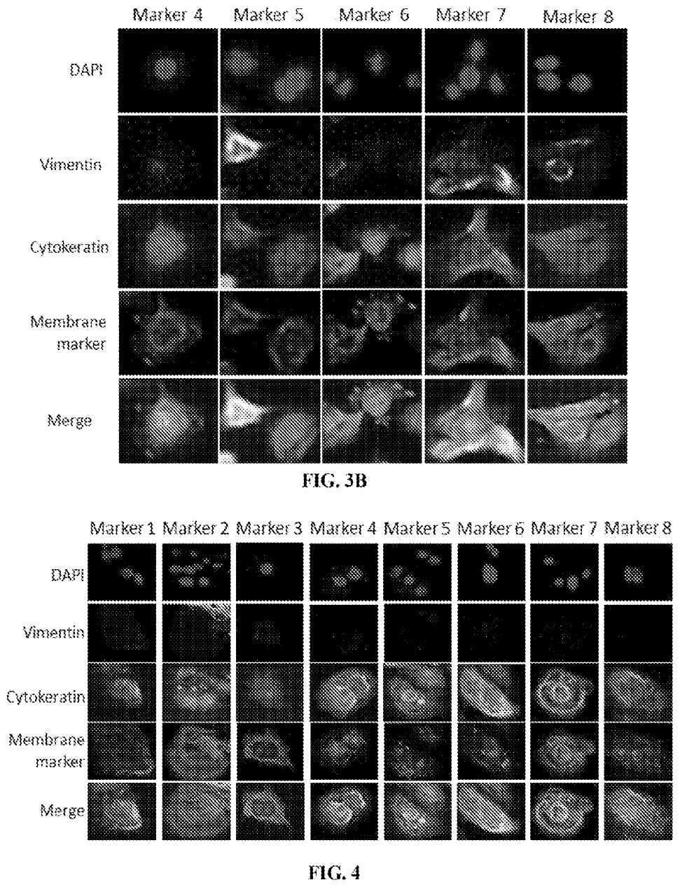

Eight different markers chosen based on RNAseq analysis were used to enrich fetal membrane cells from maternal blood. Immunofluorescence staining of the cells for these markers was performed using a pool of CK antibodies in order to localize them in fetal membrane cells. A representative staining pattern of amnion epithelial cells is shown in FIGS. 3A and 3B, and chorion cells in FIG. 4.

FIGS. 3A and 3B shows the expression of membrane specific markers in human primary amnion epithelia cells (AEC). Immunostaining shows AEC co-express epithelial marker cytokeratin-18 and mesenchymal marker vimentin, while “membrane” markers are seen on the cell and nuclear membrane and cytoplasm. FIG. 3A shows AEC express all three membrane specific markers in green, and FIG. 3B shows an additional five membrane markers in red. Fluorescent images were captured at 20×. These figures shows one representative image from three separate experiments.

FIG. 4 shows the expression of membrane specific markers in human primary chorion trophoblast (CTC). Immunostaining shows CTC dominantly express epithelial marker cytokeratin-18 (green) but not mesenchymal marker vimentin, while “membrane” markers are seen in red. Fluorescent microscopy shows CTC express all eight membrane specific markers. Fluorescent images were captured at 20×. Blue—DAPI, red—chorion membrane marker of interest, yellow—vimentin, and green—Cytokeratin-18. This figure shows one representative image from three separate experiments.

### Example 3. Rare Circulating Fetal Cells for PTB Identification

Next, it was determined whether it was feasible to enrich Circulating fetal cells (CFCs) from maternal blood in GA of 36+ weeks using antibodies for those markers that are shown to be highly expressed in the cells from the fetal membranes as compared to maternal blood cells. Briefly, 30 mL of blood was collected from pregnant women expecting a male child. Blood samples were processed using protocols taught in U.S. Pat. No. 9,429,520, and International Patent Publication No. WO2012/062325 for the isolation of fetal cells. 9 antibodies were used for the enrichment of CFCs using protocols taught in U.S. Pat. No. 9,429,520, and International Patent Publication No. WO2012/062325 for the isolation of fetal cells. The identity of some of the fetal cell candidates was confirmed by XY FISH. CFC specific antibodies were found that be used as biomarkers for (early) detection of PTB.

TABLE 5
Antibodies and markers

| Antibody | Company |
|---|---|
| MUC16, mouse | abcam ab1107 |
| UPK1B, mouse | sigmaaldrich WH0007348M2 |
| EMP1, mouse | abnova H00002012-A01 |
| GPX8, mouse | abnova H00493869-B01P |
| FLT1/VEGFR1, mouse | abcam ab212369 |
| RXFP1, mouse | rndsystems mab8898 |
| CNR1, mouse | sigma wh0001268m1 |
| PRLR, mouse | abcam u5 ab2772 |
| PRLR, mouse | abcam t6 ab2773 |

It was found that 9 different antibodies for 8 different markers expressed in both amnion and chorion from fetal cells enriched from maternal blood collected later in the pregnancy (close to term) could be used as antibodies for detection of biomarkers for detection of fetal membrane cells present in the blood of pregnant women. Since fetal membranes become fragile during parturition (term or preterm) and leads to shedding of the cells into maternal circulation, these markers may be indicative of PTB. Thus, it was shown that these 8 biomarkers may be indicative of (early) PTB.

### Example 4. Isolating Rare Circulating Fetal Cells from Samples with Pregnancy Related Complications

The present inventors determined whether shed amniochorion membrane cells can be identified in the blood of those pregnant women who have had one of the following conditions at parturition: Premature Preterm Rupture of Membranes (PPROM), Artificial Rupture of Membranes (AROM) at term, and Cesarean at term. The fetal membranes in pregnancies close to term (caesarean or normal) become fragile and hence shed cells into maternal circulation.

Methods for enriching fetal membrane cells from maternal blood can include, e.g., those taught in U.S. Pat. No. 9,429,520, and International Patent Publication No. WO2012/062325, which are directed to relevant portions incorporated herein by reference. Briefly, the method can include fixation of cells in a maternal blood sample, which greatly increases the stability of fetal cells in a maternal blood sample, while allowing enrichment and identification of fetal cells.

Blood sampling. Peripheral blood samples of 30 mL were obtained from pregnant women. All blood samples were collected in Streck tubes.

Whole blood fixation and red blood cell lysis. Blood samples were fixed in 2% formaldehyde in PBS. After lysing red blood cells, nucleated cells were harvested using methods reported previously.

Enrichment of fetal membrane cells by Magnetic-Activated Cell Sorting (MACS). Enrichment of fetal membrane cells was performed using 9 different primary antibodies targeting 8 different markers in the fetal membrane cells, mixed together according to the manufacturer's basic protocols with slight modifications. The cell suspension was incubated with the antibodies for 30 minutes, washed twice with 14 mL Magnetic Activated Cell Sorting (MACS) buffer (4° C.), recovered by centrifugation, and resuspended in MACS buffer.

After enrichment, Immunofluorescence staining of the cells was performed using a pool of CK antibodies.

Finally, fetal membrane cell candidates were identified upon scanning and validation.

From two pregnant women with PPROM, 12 fetal membrane cells were identified. A sample from a woman with AROM at term did not enrich any fetal cell. Four samples from women with Cesarean at term enriched 15 fetal cells (table 6). What is significant in the data in example 4 is that the PPROM samples (which are much earlier in gestational age render many more cells as compared to caesarean). This can only happen if the fetal membranes in PPROM are more fragile as compared to caesarean samples, thus showing that the detection of fetal cell membrane cells in the blood of pregnant women are indicative of pre-term premature rupture of the membrane leading to pre-term birth.

TABLE 6
fetal membrane cell enrichment from pregnant woman
with PPROM, AROM at term and Cesarean at term.

| | | | Average. |
|---|---|---|---|
| | No. of | | No. of |
| Sample type | samples | Total FCs. | Fetal Cells. |
| PPROM | 2 | 12 | 6 |
| AROM at term | 1 | 0 | 0 |
| Cesarean at term | 4 | 15 | 3.75 |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Richardson L S, Vargas G, Brown T, et al. Discovery and Characterization of Human Amniochorionic Membrane Microfractures. Am J Pathol. 2017; 187(12):2821-2830.”
2. Vestergaard E M, Singh R, Schelde P, et al. On the road to re-placing invasive testing with cell-based NIPT: Five clinical cases with aneuploidies, microduplication, unbalanced structural rearrangement, or mosaicism. Prenat Diagn. 2017; 37(11):1120-1124.
3. Kolvraa S, Singh R, Normand E A, et al. Genome-wide copy number analysis on DNA from fetal cells isolated from the blood of pregnant women. Prenat Diagn. 2016; 36(12):1127-1134.
4. Singh R, Hatt L, Ravn K, et al. Fetal cells in maternal blood for prenatal diagnosis: a love story rekindled. Biomark Med. 2017; 11(9):705-710.

1. An assay for identifying amnion and/or chorion cell markers in a blood sample obtained from a pregnant woman comprising:
obtaining the blood sample from the pregnant woman; and
determining the presence of one or more biomarkers selected from IGF2, IGFPB3, SERPINB10, FBN1, CRYAB, KRT18, PRTG, COL1A2, GPX8, MUC16, KRT5, TSPAN1, UPK1B, CADPS2, LAMC2, AHNAK2, EMP1, FN1, MET, PVRL4, A2ML1, DSP, THSD4, KRT17, PDLIM4, COL17A1, DKK3, PLS3, DPYSL3, TPPP3, SHROOM3, THY1, DCN, DIO2, IGFBP2, SPARCL1, NNMT, LPHN3, PEG3, FLT1, NPR3, AOC1, ITGB8, RXFP1, SPOCK1, CYP11A1, COL4A2, CNR1, SEMA3A, SERPINE1, IL1R1, FBLN1, UCHL1, RAI2, TGM2, PRLR, FSTL3, BCAR1, THSD4, FERMT2, PKP2, P4HA2, TEAD1, or AQPEP, in amnion and/or chorion cells in the blood sample or fraction thereof.

2. The assay of claim 1, wherein the presence of amnion and/or chorion cells is at least one of:
indicative of pre-term birth, an associated clinical condition, an increased risk of pre-term birth or an increased risk of an associated clinical condition of the pregnant woman, or preeclampsia; or
is determined by detecting one or more specific amnion and/or chorion cell markers.

3. (canceled)

4. The assay of claim 3, wherein the specific amnion and/or chorion cell markers are differentially expressed in amnion and/or chorion cells compared to a blood sample or a fraction thereof isolated from a pregnant woman.

5. The assay of claim 4, wherein a log 2 fold difference between the expression of the amnion and/or chorion cell marker in amnion and/or chorion cells compared with the expression in a blood sample or fraction thereof is at least 5, at least 10, or at least 15.

6. The assay according to any of the preceding claims, wherein the method comprises the steps of:
a) providing a blood sample or a fraction thereof isolated from a pregnant woman,
b) contacting the sample with (i) a ligand directed to an amnion and/or chorion cell marker or (ii) a hybridization probe comprising at least 10 contiguous nucleotides complementary to a gene encoding the amnion and/or chorion cell marker, and
c) detecting the amnion and/or chorion cell marker in the blood sample or fraction thereof of a);
wherein the presence of an amnion and/or chorion cell marker is indicative of pre-term birth or an associated clinical condition or an increased risk of preterm premature rupture of the membranes leading to pre-term birth or an associated clinical condition of the pregnant woman.

7. The assay according to claim 1, wherein the amnion and/or chorion cell marker is selected from at least one of:
the group consisting of MUC16, UPK1B, EMP1, PVRL4, THY1, DIO2, LPHN3, FLT1, NPR3, RXFP1, CNR1, PRLR, IGF2, IGFBP3, SERPINB10, FBN1, CRYAB, KRT18, PRTG, COL1A2 and GPX8;
the group consisting of MUC16, UPK1B, EMP1, PVRL4, THY1, DIO2, LPHN3, FLT1, NPR3, RXFP1, CNR, PRLR, and GPX8;
the group consisting of THY1, DIO2, LPHN3, FLT1, NPR3, RXFP1, CNR1 and PRLR; or
the group consisting of IGF2, IGFBP3, SERPINB10, FBN1, CRYAB, KRT18, PRTG, COL and GPX8.

8. (canceled)

9. (canceled)

10. (canceled)

11. (canceled)

12. The assay according to claim 1, wherein the assay further comprises detecting an epithelial marker, such as a marker selected from the group consisting of CK1, CK2, CK3, CK4, CK5, CK6, CK7, CK8, CK9, CK10, CK12, CK13, CK14, CK15, CK16, CK17, CK18 and CK19; or
the assay further comprises detecting a mesenchymal marker Vimentin.

13. (canceled)

14. The assay according to claim 1, wherein the clinical condition associated with pre-term birth is preeclampsia, pre-term birth, or a tear or rupture of the amniotic membrane.

15. The assay according to claim 1, wherein the assay further comprises:
determining the amount of amnion and/or chorion cells in the blood sample or fraction thereof, and
comparing the amount of amnion and/or chorion cells to a control;
wherein an amount of amnion and/or chorion cells in the blood sample or fraction thereof is higher than the amount in a control is indicative of preterm premature rupture of the membranes and pre-term birth or an associated clinical condition or an increased risk of pre-term birth or an associated clinical condition of the pregnant woman.

16. The assay according to claim 15, wherein the amount of amnion and/or chorion cells in a control is at least one of:
a pre-determined value;
is detected at the protein level and/or the RNA level;
is isolated from the pregnant woman after 20 weeks of gestation;
is present on the cell membrane or intracellularly.

17. (canceled)

18. (canceled)

19. (canceled)

20. (canceled)

21. The assay according to claim 1, wherein a treatment is provided to the pregnant woman for minimizing the risk of preterm birth or ameliorating the consequences of pre-term birth, such as hospitalizing the pregnant woman.

22. A method of determining preterm premature rupture of the membranes and pre-term birth or an associated clinical condition or an increased risk of pre-term birth or an associated clinical condition, the method comprising the steps of:
a) providing a blood sample or a fraction thereof isolated from a pregnant woman, and
b) determining the presence of amnion and/or chorion cells in the blood sample or fraction thereof;
wherein the presence of amnion and/or chorion cells is indicative of pre-term birth or an associated clinical condition or an increased risk of pre-term birth or an associated clinical condition of the pregnant woman.

23. The method of claim 22, wherein the presence of amnion and/or chorion cells is determined by at least one of:
detecting one or more specific amnion and/or chorion cell markers; or
differentially expressed in amnion and/or chorion cells compared to a blood sample or a fraction thereof isolated from the pregnant woman.

24. (canceled)

25. The method of claim 24, wherein the log 2 fold difference between the expression of the amnion and/or chorion cell marker in amnion and/or chorion cells compared with the expression in a blood sample or fraction thereof is at least 5, at least 10, or at least 15.

26. The method according to claim 22, wherein the method comprises the steps of:
a) contacting the sample with (i) a ligand directed to an amnion and/or chorion cell marker or (ii) a hybridization probe comprising at least 10 contiguous nucleotides complementary to a gene encoding the amnion and/or chorion cell marker, and
b) detecting the amnion and/or chorion cell marker in the blood sample or fraction thereof of a);
wherein the presence of an amnion and/or chorion cell marker is indicative of pre-term birth or an associated clinical condition or an increased risk of pre-term birth or an associated clinical condition of the pregnant woman.

27. The method according to claim 22, wherein the amnion and/or chorion cell marker is selected from at least one of:
the group consisting of IGF2, IGFPB3, SERPINB10, FBN1, CRYAB, KRT18, PRTG, COL1A2, GPX8, MUC16, KRT5, TSPAN1, UPK1B, CADPS2, LAMC2, AHNAK2, EMP1, FN1, MET, PVRL4, A2ML1, DSP, THSD4, KRT17, PDLIM4, COL17A1, DKK3, PLS3, DPYSL3, TPPP3, SHROOM3, THY1, DCN, DIO2, IGFBP2, SPARCL1, NNMT, LPHN3, PEG3, FLT1, NPR3, AOC1, ITGB8, RXFP1, SPOCK1, CYP11A1, COL4A2, CNR1, SEMA3A, SERPINE1, IL1R1, FBLN1, UCHL1, RAI2, TGM2, PRLR, FSTL3, BCAR1, THSD4, FERMT2, PKP2, P4HA2, TEAD1, and AQPEP;
the group consisting of MUC16, UPK1B, EMP1, PVRL4, THY1, DIO2, LPHN3, FLT1, NPR3, RXFP1, CNR1, PRLR, IGF2, IGFBP3, SERPINB10, FBN1, CRYAB, KRT18, PRTG, COL1A2 and GPX8;
the group consisting of MUC16, UPK1B, EMP1, PVRL4, THY1, DIO2, LPHN3, FLT1, NPR3, RXFP1, CNR, PRLR, and GPX8;
the group consisting of THY1, DIO2, LPHN3, FLT1, NPR3, RXFP1, CNR1 and PRLR;
the group consisting of IGF2, IGFBP3, SERPINB10, FBN1, CRYAB, KRT18, PRTG, COL1A2 and GPX8; or
the group consisting of MUC16, UPK1B, EMP1 and PVRL4.

28. (canceled)

29. (canceled)

30. (canceled)

31. (canceled)

32. (canceled)

33. The method according to claim 22, wherein the method further comprises detecting an epithelial marker, such as a marker selected from the group consisting of CK1, CK2, CK3, CK4, CK5, CK6, CK7, CK8, CK9, CK10, CK12, CK13, CK14, CK15, CK16, CK17, CK18 and CK19 and/or a mesenchymal marker selected from at least one of: Vimentin.

34. The method according to claim 22, wherein the method further comprises:
a) determining the amount of amnion and/or chorion cells in the blood sample or fraction thereof, and
b) comparing the amount of amnion and/or chorion cells to a pregnant women who is at a same gestational age, but not at a high risk of pre-term birth;
wherein an amount of amnion and/or chorion cells in the blood sample or fraction thereof higher than the amount in a control is indicative of pre-term birth or an associated clinical condition or an increased risk of preterm premature rupture of the membranes and pre-term birth or an associated clinical condition of the pregnant woman.

35. The method according to claim 34, wherein the amount of amnion and/or chorion cells in a control is at least one of:
a pre-determined value;
is detected at the protein level and/or the RNA level;
is isolated from the pregnant woman after 20 weeks of gestation;
is present on the cell membrane or intracellularly.

36. (canceled)

37. (canceled)

38. (canceled)

39. (canceled)

40. The method according claim 22, wherein a treatment is provided to the pregnant woman for minimizing the risk of preterm birth or ameliorating the consequences of pre-term birth, such as hospitalizing the individual.

41. A kit comprising one or more agents for the detection of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 49, 49, 50, 51, 52, 53, 54, 55, 56, 57, 58, 59, 60, 61, 62, 63 or all the markers in Table 1 and/or table 2, on amnion and/or chorion cells in a blood sample or fraction thereof.

42. A method of determining preterm premature rupture of the membranes and pre-term birth or an associated clinical condition or an increased risk of pre-term birth or an associated clinical condition, the method comprising the steps of:
a) obtaining a blood sample or a fraction thereof isolated from a pregnant woman, and
b) determining the presence of amnion and/or chorion cells in the blood sample or fraction thereof;
c) isolating the amnion and/or chorion cells in the blood sample or fraction thereof;
d) determining the RNA sequence of the isolated amnion/chorion cells;
e) comparing a gene expression profile of the isolated amnion/chorion cells to a pregnant women who is at a same gestational age, but not at a high risk of pre-term birth;
wherein the gene expression profile is indicative of pre-term birth or an associated clinical condition or an increased risk of pre-term birth or an associated clinical condition of the pregnant woman.

43. The method according to claim 42, wherein the clinical condition associated with pre-term birth is preeclampsia.

44. The method according to claim 42, wherein the method further comprises detecting a maternal marker, such as a marker selected from the group consisting of CD14 and CD45.

45. Amnion and/or chorion specific markers for use in diagnosing risk of premature birth.
